(19) United States
(12) Patent Application Publication
(10) Pub. No.: US 2021/0222225 A1
(43) Pub. Date: Jul. 22, 2021

(54) Medium for Disk Diffusion Method to Evaluate Antibiotic Sensitivity

(71) Applicant: INDUSTRY-ACADEMIC COOPERATION FOUNDATION, YONSEI UNIVERSITY, Seoul (KR)

(72) Inventors: Dong Eun Yong, Seoul (KR); Jean Damascene Uwizeyimana, Kigali City (RW); Jung Hyun Byun, Seoul (KR)

(21) Appl. No.: 17/257,493

(22) PCT Filed: Jul. 4, 2019
(86) PCT No.: PCT/KR2019/008241

§ 371 Date: Dec. 31, 2020

(30) Foreign Application Priority Data
Jul. 4, 2018 (KR) 10-2018-0077796

(51) Int. Cl. C12Q 1/18

(52) U.S. Cl. CPC C12Q 1/18

(57) ABSTRACT

The present invention relates to a medium composition that can be used in a disk diffusion method to evaluate antibiotic susceptibility, and more specifically to a medium composition for a disk diffusion method, comprising 1.5 to 10 g/L of agar; and at least one of 50 to 200 μg/ml of protamine or a salt thereof and 5 to 500 μg/ml of guanidine or a salt thereof.

### CROSS-REFERENCE TO RELATED APPLICATIONS

This application is a U.S. National Stage entry of International Patent Application no. PCT/KR2019/008241, filed Jul. 4, 2019, which claims the benefit of priority of Korean Patent Application no. 10-2018-0077796, filed Jul. 4, 2018.

### TECHNICAL FIELD

The present invention relates to a medium composition that can be used in a disk diffusion method to evaluate antibiotic susceptibility, and to a method for evaluating antibiotic susceptibility by a disk diffusion method using the same.

### BACKGROUND ART

In general, a colony counting method and a disk diffusion method are used to measure antimicrobial activity of a specimen. The colony counting method is a method in which a microbial solution that has reacted with a specimen for a certain period of time is plated on nutrient agar, the microorganisms therein are allowed to grow for a certain period of time, and the grown microbial colonies are counted to measure antimicrobial activity of the specimen. This method makes it possible to obtain a quantitative measurement result.

On the other hand, the disk diffusion method is a method in which a specimen is placed on nutrient agar on which a certain amount of microorganisms have been plated, the microorganisms are allowed to grow for a certain period of time, and a size of a microbial growth inhibition zone, which is formed around the specimen under the specimen's influence, is measured. This method makes it possible to obtain a qualitative antimicrobial activity result for a specimen.

Specifically, the colony counting method is widely used in various research fields in that it is possible to obtain a quantitative antimicrobial activity measurement result. However, this method is disadvantageous in terms of time and cost from the viewpoints that to obtain a measurable microbial colony count, repeated experiments using various dilution ratios are required, and a time during which microorganisms are allowed to react with a specimen and a time during which the microorganisms after reaction are allowed to grow on nutrient agar are respectively required.

On the other hand, the disk diffusion method is advantageous, in terms of test time and test cost, as compared with the colony counting method, from the viewpoint that microorganisms in a certain amount are plated on nutrient agar, and then the microorganisms are allowed to grow with a specimen placed thereon so that reaction between the microorganisms and the specimen occurs along with growth of the microorganisms. This experiment is mainly used exclusively for studies to obtain a minimum inhibitory concentration (MIC) of an antimicrobial agent required to inhibit microbial growth.

### Technical Problem

An object of the present invention is to provide a medium composition that can be used in a disk diffusion method to evaluate antibiotic susceptibility.

Another object of the present invention is to provide a method for evaluating antibiotic susceptibility by a disk diffusion method using the medium composition of the present invention.

However, the technical problem to be achieved by the present invention is not limited to the above-mentioned problem, and other problems that are not mentioned will be clearly understood by those of ordinary skill in the art from the following description.

### Solution to Problem

A recent global challenge is to develop new antibiotics in development of drugs for the treatment of multi-drug resistant (MDR) gram-negative (GN) bacteria. On the other hand, colistin corresponds to a last-resort antibiotic means for managing the MDR GN bacteria. Colistin, known as polymyxin E, corresponds to one of cationic, cyclic polypeptide antibiotics. This colistin was first isolated from Paenibacillus polymyxa subsp, which corresponds to a soil bacterium, in Japan in 1947, and then was used for clinical use in 1959. However, due to risk of nephrotoxicity and neurotoxicity, its use has been discontinued since the 1980s.

However, several recent studies have shown that intravenous injection of colistin sulfate is safe and can manage severe infections of MDR GN pathogens. Thus, from the mid-1990s, polymyxins were again used clinically. Among these, colistin is attracting attention due to its rapid bactericidal effect, its broad spectrum of action against MDR GN pathogens, and lack of new antibiotics against most MDR GN bacteria. Colistin exhibits specific activity against MDR strains such as Klebsiella spp., Enterobacter spp., Escherichia coli, Citrobacter spp., Salmonella spp., and Shigella spp. In addition, colistin exhibits activity against GN bacteria, such as Stenotrophomonas maltophilia, Acinetobacter baumannii, and Pseudomonas aeruginosa, while showing a low level of resistance. In general, a global rate of resistance to polymyxins is lower than 10% and is on an increasing trend.

Despite a fairly long clinical use of colistin, it still remains a challenge to find an appropriate method capable of evaluating susceptibility to colistin. With a rapidly increased use of colistin over the past few years, there is a growing demand for a faster, more accurate, and more reliable in vitro antibiotic susceptibility testing (AST) method that allows clinicians to make a therapeutically appropriate decision.

A disk diffusion method is commonly used in many clinical laboratories. However, this method is problematic in that accuracy in susceptibility evaluation is very poor due to decreased diffusion capability of colistin to medium, as compared with a MIC-based method. Other methods used include Vitek2, E-test, agar dilution, and the like, and these are also disadvantageous due to their poor reliability. Since colistin AST has already been standardized and used in clinical microbiology laboratories, it is difficult for researchers to change conditions under which a disk diffusion method is performed.

As a result of conducting continuous research to solve the above-mentioned problems, the present inventors have found composition of medium that can increase accuracy of a disk diffusion method for predicting susceptibility to polymyxins, in particular, colistin, and thus have completed the present invention.

According to an embodiment of the present invention, there is provided a medium composition for a disk diffusion method, comprising 1.5 to 10 g/L of agar; and at least one of 50 to 200 μg/ml of protamine or a salt thereof and 5 to 500 μg/ml of guanidine or a salt thereof.

In the present invention, the medium composition may be a medium composition for a disk diffusion method for evaluating antibiotic susceptibility.

In the present invention, the antibiotic may be a polymyxin, and more preferably colistin. In the present invention, the “polymyxin” is an antibiotic for treating Gram-negative bacterial infections, and is produced from Gram-positive bacteria such as Paenibacillus polymyxa. This polymyxin binds to lipopolysaccharide on the outer cell membrane of Gram-negative bacteria and then destroys the outer and inner cell membranes, thereby exerting an antibiotic effect.

In the medium composition of the present invention, the agar may be contained in an amount of 1.5 to 10 g/L, and preferably 1.7 to 8.5 g/L, 3.4 to 6.8 g/L, 4.0 to 6.0 g/L, or 4.5 to 5.5 g/L. In the present invention, in a case where a content of the agar is lower than 1.5 g/L, the medium is not sufficiently solidified and thus is not suitable for a disk diffusion method; and in a case where a content of the agar exceeds 10 g/L, an antibiotic fails to diffuse into the agar and thus a small growth inhibition zone is formed, so that accuracy in antibiotic susceptibility evaluation is remarkably decreased.

In addition, the medium composition of the present invention may comprise at least one of protamine or a salt thereof and guanidine or a salt thereof.

In the present invention, the salt of protamine may be, but is not limited to, protamine sulfate.

In the present invention, the salt of guanidine is preferably guanidine hydrochloride.

In the medium composition of the present invention, the protamine or a salt thereof may be contained in an amount of 50 to 200 μg/ml, preferably 50 to 150 μg/ml, and more preferably 80 to 120 μg/ml. In the present invention, in a case where a content of the protamine or a salt thereof is lower than 50 μg/ml, an effect of enhancing diffusion capability of an antibiotic is insignificant; and in a case where a content of the protamine or a salt thereof exceeds 200 μg/ml, bacteria are inhibited in their growth or killed due to the protamine or a salt thereof, so that accuracy in antibiotic susceptibility evaluation is remarkably decreased.

In the medium composition of the present invention, the guanidine or a salt thereof may be contained in an amount of 5 to 500 μg/ml, and preferably in an amount of 10 to 300 μg/ml or 25 to 100 μg/ml. In the present invention, in a case where a content of the guanidine or a salt thereof is lower than 5 μg/ml, an effect of enhancing diffusion capability of an antibiotic is insignificant; and in a case where a content of the guanidine or a salt thereof exceeds 500 μg/ml, bacteria are inhibited in their growth or killed due to the guanidine or a salt thereof, so that accuracy in antibiotic susceptibility evaluation is remarkably decreased.

The medium composition of the present invention may further comprise a beef extract in an amount of 1.5 to 2.5 g/L and preferably 1.75 to 2.25 g/L.

The medium composition of the present invention may further comprise a casein hydrolysate in an amount of 15 to 20 g/L and preferably 17 to 18 g/L.

The medium composition of the present invention may further comprise starch in an amount of 1 to 2 g/L and preferably 1.25 to 1.75 g/L.

The medium composition of the present invention may further comprise a residual solvent (preferably water).

Preferably, in the present invention, the medium composition may comprise 1.5 to 10 g/L of agar; at least one of 50 to 200 μg/ml of protamine or a salt thereof and 5 to 500 μg/ml of guanidine or a salt thereof; 1.5 to 2.5 g/L of a beef extract; 15 to 20 g/L of a casein hydrolysate; 1 to 2 g/L of starch; and residual water.

The medium composition of the present invention may have a pH of 7 to 7.5, and preferably 7.2 to 7.4, at room temperature of 25° C. However, the present invention is not limited thereto.

According to another embodiment of the present invention, there is provided a method for evaluating antibiotic susceptibility, comprising steps of: plating a desired strain on the medium composition of the present invention; and placing an antibiotic-containing specimen on the strain-plated medium composition.

In the present invention, the medium composition may be prepared by being supported on a culture plate. In the present invention, the culture plate is not particularly limited in material and shape, and may be, for example, a circular plate made of polystyrene or glass.

In the present invention, the desired strain is a strain used to predict antibiotic susceptibility, and may be Gram-positive bacteria or Gram-negative bacteria. However, specific types of the strain are not particularly limited.

In the present invention, the specimen is not particularly limited in material, size, and shape. For example, the specimen may be made of paper or cellulose material and may be in a disk shape having a diameter of 5 to 20 mm. In the present invention, for the specimen, a commercially available disk, that is, a Whatman paper disk (Whatman Ltd.) having a diameter of 6 mm, 9 mm, or 13 mm, or a blank disk (Oxoid) may be used. However, the present invention is not limited thereto.

In the present invention, the specimen may be immersed in a solution containing an antibiotic and a solvent.

In the present invention, the antibiotic may be a polymyxin, and more preferably colistin.

In the present invention, the solvent may be, but is not limited to, one or more selected from the group consisting of purified water, methanol, ethanol, glycerin, ethyl acetate, butylene glycol, propylene glycol, dichloromethane, chloroform, ethyl ether, butylene glycol, hexane, and mixtures thereof.

In the present invention, an antibiotic concentration in the solution is not particularly limited, and may vary depending on type of antibiotic used and method of determining antibiotic susceptibility. For example, the antibiotic may be contained in an amount of 0.1 to 1,000 μg/ml or 0.5 to 100 μg/ml.

In the present invention, the specimen may be immersed in the solution and maintained, at room temperature of 20° C. to 25° C., for 10 minutes or longer, 15 minutes or longer, 30 minutes or longer, 1 hour or longer, 2 hours or longer, 4 hours or longer, 6 hours or longer, 12 hours or longer, 1 day or longer, 2 days or longer, 4 days or longer, 8 days or longer, 15 days or longer, or 30 days or longer.

In the present invention, a step of drying the solution-immersed specimen may be performed at room temperature for 10 minutes or longer, 15 minutes or longer, 30 minutes or longer, 1 hour or longer, 2 hours or longer, 4 hours or longer, 6 hours or longer, 12 hours or longer, 14 hours or longer, 16 hours or longer, 18 hours or longer, 20 hours or longer, 22 hours or longer, or 24 hours or longer.

In the present invention, after the step of placing the specimen on the medium composition, if necessary, a step of maintaining the specimen at 4° C. for 1 minute to 24 hours, 10 minutes to 12 hours, 1 to 6 hours, or 1 to 3 hours may be performed for pre-diffusion of the antibiotic.

In addition, in the present invention, after placing the specimen on the medium composition, a step of culturing the desired strain may be performed. Here, conditions for the culture are not particularly limited, and may be appropriately regulated depending on type of the desired strain. For example, the culture may be performed at 5 vol % CO₂ and 37° C. for 10 minutes or longer, 15 minutes or longer, 30 minutes or longer, 1 hour or longer, 2 hours or longer, 4 hours or longer, 6 hours or longer, 12 hours or longer, 14 hours or longer, 16 hours or longer, 18 hours or longer, 20 hours or longer, 22 hours or longer, 24 hours or longer, 48 hours or longer, 56 hours or longer, 64 hours or longer, 72 hours or longer, or 80 hours or longer.

In the present invention, after the step of culturing the strain, the method may further comprise a step of measuring a size, preferably a diameter, of a microbial growth inhibition zone formed around the specimen, to determine antibiotic susceptibility of the desired strain.

In the present invention, the “growth inhibition zone” corresponds to a clear region that is formed around the specimen (that is, a paper disk) due to inhibition of bacterial growth caused by the antibiotic diffused from the disk. In general, a size of the growth inhibition zone is directly related to bacterial antibiotic susceptibility: This means that a larger growth inhibition zone indicates greater bacterial antibiotic susceptibility. However, the size of the growth inhibition zone may vary depending on type of the strain, amount of the antibiotic deposited on the disk, and culture conditions.

In the present invention, the antibiotic to be measured for susceptibility may be a polymyxin, and more preferably colistin.

### Advantageous Effects of Invention

In a case where a medium composition provided in the present invention is used, when determining susceptibility to an antibiotic, preferably a polymyxin, and more preferably colistin using a disk diffusion method, it is possible to increase diffusion capability of the antibiotic from a disk to medium, thereby further increasing accuracy in susceptibility measurement.

### BRIEF DESCRIPTION OF DRAWINGS

FIG. 1 illustrates a diameter size (mm) of a growth inhibition zone spread from a disk, observed after each of colistin-susceptible (S) or -resistant (R) Acinetobacter (ACB) strain, Pseudomonas aeruginosa ATCC27853 strain, and mcr-1-harboring Enterobacter aerogenes strain is cultured in normal Mueller-Hinton agar medium; medium obtained by adding, to normal Muller-Hinton agar medium, protamine at a concentration of 100 μg/ml; Muller-Hinton agar medium obtained by adjusting agar to a concentration of 5.1 g/L (30%); Muller-Hinton agar medium obtained by adjusting agar to a concentration of 5.1 g/L (30%) and then adding protamine at a concentration of 100 μg/ml; and Muller-Hinton agar medium obtained by adjusting agar to a concentration of 5.1 g/L (30%) and then adding protamine at a concentration of 150 μg/ml, in Example 1 of the present invention.

FIG. 2A illustrates a diameter size (mm) of a growth inhibition zone spread from a disk, observed after each of colistin-susceptible (S) or -resistant (R) Acinetobacter (ACB) strain (N=2), Pseudomonas aeruginosa ATCC27853 strain, and mcr-1-harboring Enterobacter aerogenes strain is cultured in a medium composition containing agar at a concentration of 17 g/L (100%) and protamine at a concentration of 100 μg/ml, in Example 4 of the present invention.

FIG. 2B illustrates a diameter size (mm) of a growth inhibition zone spread from a disk, observed after each of colistin-susceptible (S) or -resistant (R) Acinetobacter (ACB) strain (N=2), Pseudomonas aeruginosa ATCC27853 strain, and mcr-1-harboring Enterobacter aerogenes strain is cultured in a medium composition containing agar at a concentration of 5.1 g/L (30%) and protamine at a concentration of 100 μg/ml, in Example 4 of the present invention.

FIG. 3A illustrates a picture of a growth inhibition zone spread from a disk, observed after colistin-susceptible Pseudomonas aeruginosa ATCC27853 is cultured in a medium composition containing agar at a concentration of 17 g/L (100%) and protamine at a concentration of 100 μg/ml, in Example 4 of the present invention.

FIG. 3B illustrates a picture of a growth inhibition zone spread from a disk, observed after colistin-susceptible Pseudomonas aeruginosa ATCC27853 is cultured in a medium composition containing agar at a concentration of 5.1 g/L (30%) and protamine at a concentration of 100 μg/ml, in Example 4 of the present invention.

FIG. 4A illustrates a picture of a growth inhibition zone spread from a disk, observed after colistin-resistant mcr-1-harboring Enterobacter aerogenes is cultured in a medium composition containing agar at a concentration of 17 g/L (100%) and protamine at a concentration of 100 μg/ml, in Example 4 of the present invention.

FIG. 4B illustrates a picture of a growth inhibition zone spread from a disk, observed after colistin-resistant mcr-1-harboring Enterobacter aerogenes is cultured in a medium composition containing agar at a concentration of 5.1 g/L (30%) and protamine at a concentration of 100 μg/ml, in Example 4 of the present invention.

FIG. 5A illustrates a picture of a growth inhibition zone spread from a disk, observed after colistin-resistant Acinetobacter (ACB) is cultured in a medium composition containing agar at a concentration of 17 g/L (100%) and protamine at a concentration of 100 μg/ml, in Example 4 of the present invention.

FIG. 5B illustrates a picture of a growth inhibition zone spread from a disk, observed after colistin-resistant Acinetobacter (ACB) is cultured in a medium composition containing agar at a concentration of 5.1 g/L (30%) and protamine at a concentration of 100 μg/ml, in Example 4 of the present invention.

FIG. 6A illustrates a picture of a growth inhibition zone spread from a disk, observed after colistin-susceptible Acinetobacter (ACB) is cultured in a medium composition containing agar at a concentration of 17 g/L (100%) and protamine at a concentration of 100 μg/ml, in Example 4 of the present invention.

FIG. 6B illustrates a picture of a growth inhibition zone spread from a disk, observed after colistin-susceptible Acinetobacter (ACB) is cultured in a medium composition containing agar at a concentration of 5.1 g/L (30%) and protamine at a concentration of 100 μg/ml, in Example 4 of the present invention.

FIGS. 7A, 7B, 7C, 7D, 7E and 7F illustrate graphs, comparing disk diffusion method results obtained by using the medium compositions of the present invention with MIC results, in Example 4 of the present invention.

FIG. 8 illustrates pictures of a growth inhibition zone spread from a disk, observed after each of colistin-susceptible (S) or -resistant (R) Acinetobacter baumanni (ABA) strain, colistin-susceptible (S) or -resistant (R) Klebsiella pnemoniae (KPN) strain, and colistin-susceptible (S) or -resistant (R) Pseudomonas aeruginosa (PAE) strain is cultured in a medium composition containing agar at a concentration of 5.1 g/L (30%) and guanidine hydrochloride at a concentration of 500 μg/ml, in Example 5 of the present invention.

FIG. 9 illustrates pictures of a growth inhibition zone spread from a disk, observed after each of colistin-susceptible (S) or -resistant (R) Acinetobacter baumanni (ABA) strain, colistin-susceptible (S) or -resistant (R) Klebsiella pnemoniae (KPN) strain, and colistin-susceptible (S) or -resistant (R) Pseudomonas aeruginosa (PAE) strain is cultured in a medium composition containing agar at a concentration of 5.1 g/L (30%) and guanidine hydrochloride at a concentration of 50 μg/ml, in Example 5 of the present invention.

FIG. 10 illustrates pictures of a growth inhibition zone spread from a disk, observed after each of colistin-susceptible (S) or -resistant (R) Acinetobacter baumanni (ABA) strain, colistin-susceptible (S) or -resistant (R) Klebsiella pnemoniae (KPN) strain, and colistin-susceptible (S) or -resistant (R) Pseudomonas aeruginosa (PAE) strain is cultured in a medium composition containing agar at a concentration of 5.1 g/L (30%) and guanidine hydrochloride at a concentration of 5 μg/ml, in Example 5 of the present invention.

### DETAILED DESCRIPTION OF INVENTION

According to an embodiment of the present invention, there is provided a medium composition for a disk diffusion method, comprising 1.5 to 10 g/L of agar; and at least one of 50 to 200 μg/ml of protamine or a salt thereof and 5 to 500 μg/ml of guanidine or a salt thereof.

In the present invention, the medium composition may be a medium composition for a disk diffusion method for evaluating antibiotic susceptibility.

According to another embodiment of the present invention, there is provided a method for evaluating antibiotic susceptibility, comprising steps of: plating a desired strain on the medium composition of the present invention; and placing an antibiotic-containing specimen on the strain-plated medium composition.

In the present invention, the antibiotic may be a polymyxin, and more preferably colistin.

Hereinafter, the present invention will be described in more detail by way of examples. These examples are only for describing the present invention in more detail, and it will be apparent to those of ordinary skill in the art that according to the gist of the present invention, the scope of the present invention is not limited by these examples.

### EXAMPLES

### Example 1

Modification of MHA medium was performed while decreasing a concentration of agar particles (BD Diagnostic, Sparks, Md., USA) in Mueller-Hinton broth medium (BD Diagnostic), in which starting from 100% that is 17 g/L, the concentration was gradually decreased to 30% by 10%. To each MHA thus modified was added protamine (Sigma-Aldrich, St Louis, Mo., USA) at a concentration of 100 μg/ml, and solidification was performed. Then, on a surface of the medium was evenly inoculated each of colistin-susceptible (S) or -resistant (R) Acinetobacter calcoaceticus-baumannii complex (ACB) strain, Pseudomonas aeruginosa ATCC27853 strain, Escherichia coli ATCC25922 strain, and mcr-1-harboring Enterobacter aerogenes strain, and a colistin disk was placed thereon. Subsequently, a diameter of a growth inhibition zone (colistin inhibition zone), which had been formed clear by diffusion of colistin to the agar around the colistin disk, was measured. The results are shown in Table 1 below.

TABLE 1

| | | Diameter (mm) of colistin inhibition zone | | | | | | | |
|---|---|---|---|---|---|---|---|---|---|
| | | 100% | 90% | 80% | 70% | 60% | 50% | 40% | 30% |
| Strain | | agar | agar | agar | agar | agar | agar | agar | agar |
| Colistin- | ACB-R | 11 | 12 | 12 | 11 | 13 | 10 | 11 | 11 |
| resistant | mcr-1 | 10 | 10 | 9 | 10 | 10 | 11 | 10 | 11 |
| Colistin- | ACB-S | 12 | 12 | 12 | 13 | 13 | 13 | 15 | 16 |
| suscept- | PAE | 13 | 13 | 14 | 14 | 15 | 14 | 15 | 16 |
| ible | ECO | 13 | 13 | 14 | 14 | 14 | 14 | 14 | 16 |

| | | | | | | | | |
|---|---|---|---|---|---|---|---|---|
| Average | 13 | 13 | 13 | 14 | 14 | 14 | 15 | 16 |
| Difference | 2 | 1 | 1 | 3 | 1 | 4 | 4 | 5 |
| (S vs ACB-R) | | | | | | | | |
| Difference | 3 | 3 | 4 | 4 | 4 | 3 | 5 | 5 |
| (S vs mcr-1) | | | | | | | | |

As shown in Table 1, it was found that as compared with a case where the agar was added at a concentration of 17 g/L, in a case where the agar concentration was adjusted to 5.1 to 8.5 g/L, which is a concentration range equivalent to 30% to 50% of 17 g/L, a size of a growth inhibition zone in the colistin-resistant strains was maintained, and a size of a growth inhibition zone in the colistin-susceptible strains was greatly increased.

### Example 2

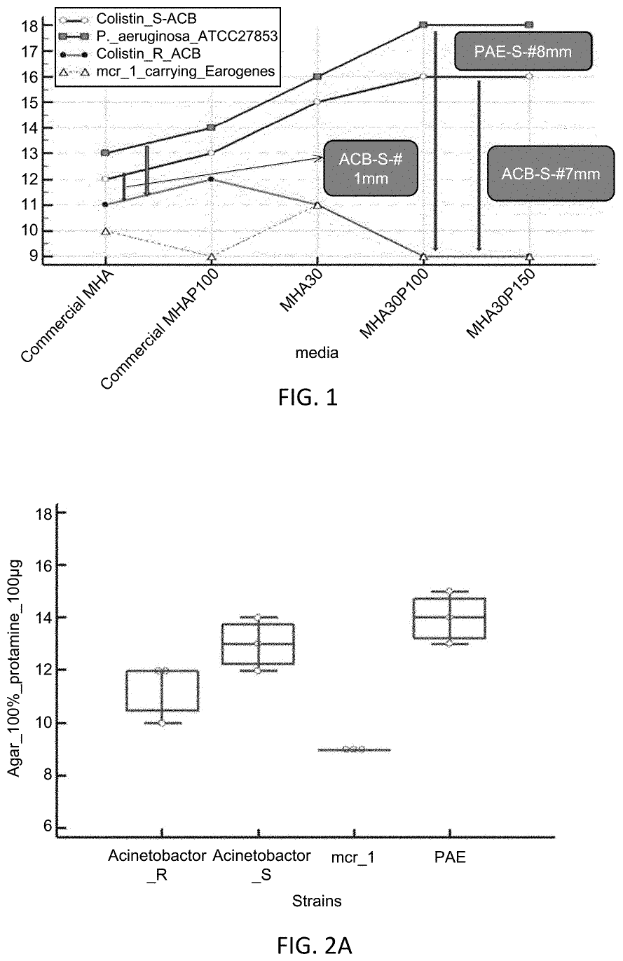

As in Example 1, a concentration of agar particles (BD Diagnostic, Sparks, Md., USA) in Mueller-Hinton broth medium (BD Diagnostic) was adjusted to 5.1 g/L, which is 30% of 17 g/L, and protamine (Sigma-Aldrich, St Louis, Mo., USA) was added thereto at a concentration of 150 μg/ml or 100 μg/ml. Then, solidification was performed. To compare effects depending on concentrations of agar and protamine, Muller-Hinton agar medium (BD Diagnostic) containing agar at a concentration of 17 g/L and medium obtained by adding, to the Muller-Hinton agar medium, protamine at a concentration of 100 μg/ml were prepared as controls. On a surface of each medium thus prepared was evenly inoculated each of colistin-susceptible (S) or -resistant (R) Acinetobacter calcoaceticus-baumannii complex (ACB) strain, Pseudomonas aeruginosa ATCC27853 strain, and mcr-1-harboring Enterobacter aerogenes strain, and a colistin disk was placed thereon. Then, it was determined whether each strain was grown, and a diameter of a growth inhibition zone, which had been formed clear around the disk, was measured. The results are illustrated in FIG. 1.

As illustrated in FIG. 1, it was found that as compared with a case where the agar was added at a concentration of 17 g/L, in a case where the agar concentration was adjusted to 5.1 g/L, which is a concentration equivalent to 30% of 17 g/L, a size of a growth inhibition zone in the colistin-resistant strains was maintained, and a size of a growth inhibition zone in the colistin-susceptible strains was increased. In addition, it was found that the above-mentioned effect was further increased in a case where protamine was added.

### Example 3

As in Example 1, a concentration of agar particles (BD Diagnostic, Sparks, Md., USA) in Mueller-Hinton broth medium (BD Diagnostic) was adjusted to 17 g/L or 5.1 g/L that is 30% of 17 g/L. To select a concentration of protamine, which can enhance diffusion of colistin from a disk without interfering with bacterial growth, while using the protamine at various concentrations, protamine (Sigma-Aldrich, St Louis, Mo., USA) was added to the medium at concentrations of 1 mg/ml, 700 μg/ml, 400 μg/ml, 300 μg/ml, 200 μg/ml, 150 μg/ml, 100 μg/ml, and 50 μg/ml. Each medium thus modified was solidified. Then, on a surface of each medium was evenly inoculated each of colistin-susceptible (S) or -resistant (R) Acinetobacter calcoaceticus-baumannii complex (ACB) strain, Pseudomonas aeruginosa ATCC27853 strain, and mcr-1-harboring Enterobacter aerogenes strain, and a colistin disk was placed thereon. Then, it was determined whether each strain was grown, and a diameter of a growth inhibition zone, which had been formed around the disk, was measured. The results are illustrated in Table 2 below.

TABLE 2

| | | | Colistin-resistant | | Colistin-susceptible | | | | | |
|---|---|---|---|---|---|---|---|---|---|---|
| | | | Acinetobacter | | Acinetobacter | | PAE ATCC 27853 | | mcr-1 | |
| | | | | Disk | | Disk | | Disk | | Disk |
| Agar | Protamine | | | diffusion | | diffusion | | diffusion | | diffusion |
| concentration | concentration | | Growth | (mm) | Growth | (mm) | Growth | (mm) | Growth | (mm) |
| 100% | 1,000 | μg/ml | Not | 6 | Not | 6 | Partially | 18 | Not | 6 |
| | | | grown | | grown | | grown | | grown | |
| | 700 | μg/ml | Not | 6 | Grown | 10 | Partially | 17 | Grown | 10 |
| | | | grown | | | | grown | | | |
| | 400 | μg/ml | Grown | 13 | Grown | 14 | Grown | 15 | Grown | 8 |
| | 300 | μg/ml | Grown | 12 | Grown | 13 | Grown | 14 | Grown | 10 |
| | 200 | μg/ml | Grown | 12 | Grown | 12 | Grown | 13 | Grown | 9 |
| | 150 | μg/ml | Grown | 13 | Grown | 13 | Grown | 14 | Grown | 10 |
| | 100 | μg/ml | Grown | 12 | Grown | 12 | Grown | 14 | Grown | 9 |
| | 50 | μg/ml | Grown | 13 | Grown | 13 | Grown | 14 | Grown | 9 |
| 30% | 1,000 | μg/ml | Not | 6 | Not | 6 | Not | 6 | Not | 6 |
| | | | grown | | grown | | grown | | grown | |
| | 700 | μg/ml | Not | 6 | Not | 6 | Not | 6 | Not | 6 |
| | | | grown | | grown | | grown | | grown | |
| | 400 | μg/ml | Not | 6 | Not | 6 | Not | 6 | Not | 6 |
| | | | grown | | grown | | grown | | grown | |
| | 300 | μg/ml | Not | 6 | Not | 6 | Not | 6 | Not | 6 |
| | | | grown | | grown | | grown | | grown | |
| | 200 | μg/ml | Partially | 8 | Partially | 14 | Grown | 20 | Grown | 8 |
| | | | grown | | grown | | | | | |
| | 150 | μg/ml | Grown | 10 | Grown | 15 | Grown | 18 | Grown | 9 |
| | 100 | μg/ml | Grown | 11 | Grown | 15 | Grown | 18 | Grown | 9 |
| | 50 | μg/ml | Grown | 13 | Grown | 14 | Grown | 17 | Grown | 9 |

Even in Table 2, it was found that as compared with a case where the agar was added at a concentration of 17 g/L, in a case where the agar concentration was adjusted to 5.1 g/L, which is a concentration equivalent to 30% of 17 g/L, a size of a growth inhibition zone in the colistin-resistant strains was maintained, and a size of a growth inhibition zone in the colistin-susceptible strains was greatly increased.

In addition, it was found that in a case where the protamine was added at a concentration of 50 to 200 μg/ml and preferably 50 to 150 μg/ml, a size of a growth inhibition zone was increased specifically in the colistin-susceptible strains, without interfering growth of each strain.

### Example 4

Step I

1. Determination of Agar Concentration to Discriminate Colistin Susceptibility in Clinical Isolates Including Mcr-1 Producers and Titration Through Addition of Protamine

The following four bacterial strains were used to determine conditions for agar: colistin-susceptible (S) or -resistant (R) Acinetobacter calcoaceticus-baumannii complex (ACB) strain (N=2), Pseudomonas aeruginosa ATCC27853 strain, and mcr-1-harboring Enterobacter aerogenes strain. A concentration of agar particles (BD Diagnostic, Sparks, Md., USA) in Mueller-Hinton broth medium (BD Diagnostic) was adjusted to 17 g/L or 5.1 g/L that is 30% of 17 g/L. To each modified MHA was added protamine (Sigma-Aldrich, St Louis, Mo., USA) at a concentration of 100 μg/ml.

2. Results

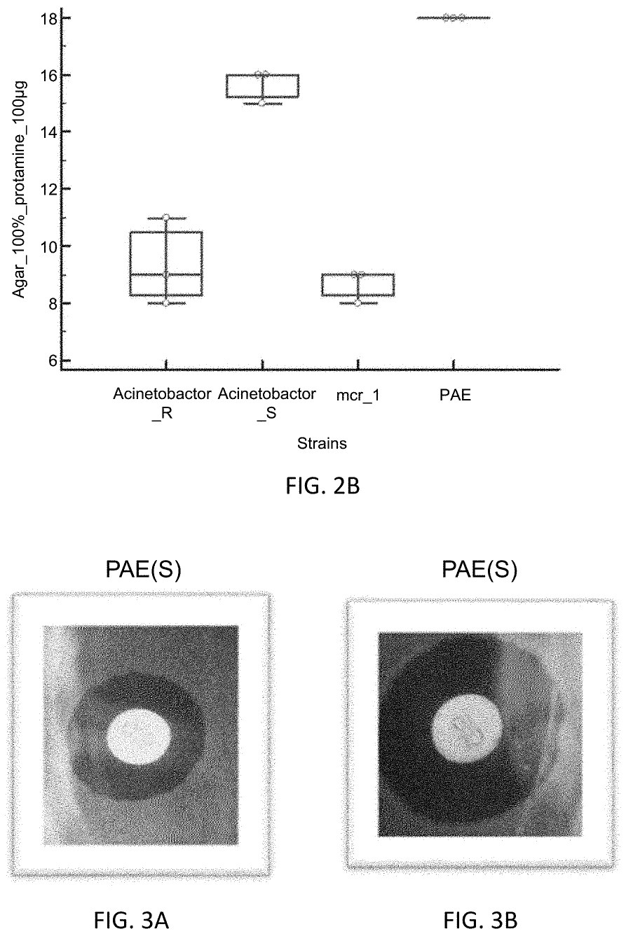

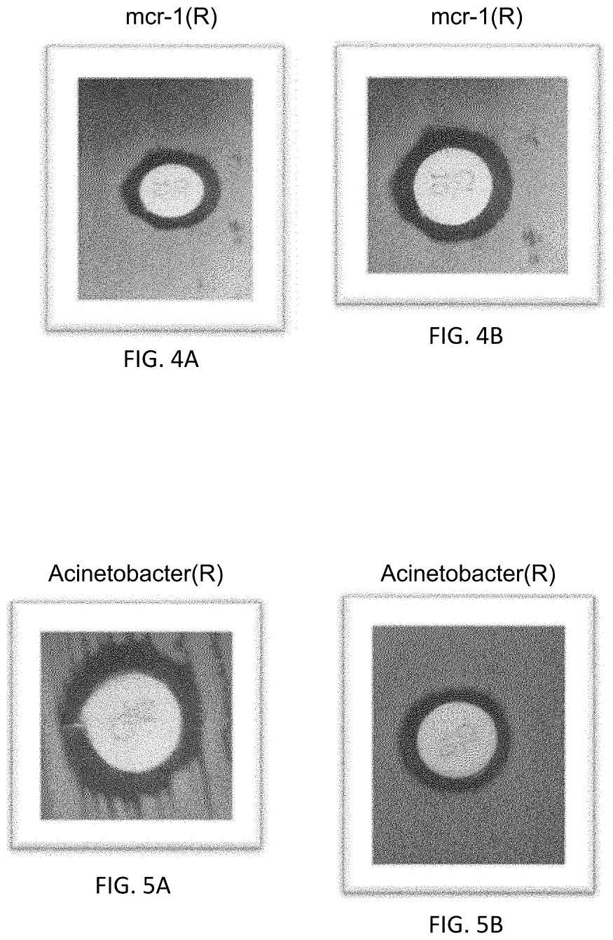

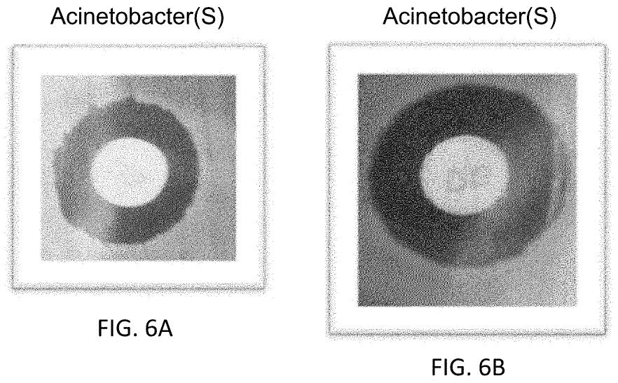

On a surface of each medium thus prepared was evenly inoculated each of colistin-susceptible (S) or -resistant (R) Acinetobacter calcoaceticus-baumannii complex (ACB) strain, Pseudomonas aeruginosa ATCC27853 strain, and mcr-1-harboring Enterobacter aerogenes strain, and a colistin disk was placed thereon. Then, it was determined whether each strain was grown, and a diameter of a colistin inhibition zone, which had been formed clear around the disk, was measured. The results are illustrated in FIGS. 2A and 2B. Pictures of an inhibition zone around the disk were taken for each strain, and the results are illustrated in FIGS. 3A, 3B, 4A, 4B, 5A, 5B, 6A and 6B.

As illustrated in FIGS. 2A and 2B and FIGS. 3A, 3B, 4A, 4B, 5A, 5B, 6A and 6B, it was found that as compared with a case where the agar concentration was adjusted to 17 g/L, in a case where the agar concentration was adjusted to 5.1 g/L and the protamine was added at a concentration of 100 μg/ml, no changes in size of the growth inhibition zone were observed in the colistin-resistant strains, and the diameter of the growth inhibition zone in the colistin-susceptible strains was remarkably increased. From these results, the agar concentration was determined to be 5.1 g/L, which is a concentration equivalent to 30% of 17 g/L contained in normal Muller-Hinton medium.

Step II

1. Determination of Correlation Between Diameter of Inhibition Zone in Modified Mueller-Hinton Medium and MIC

Experiments were performed using 63-GN clinical isolates, including P. aeruginosa (n=27), ACB (n=33), Escherichia coli ATCC25922, P. aeruginosa ATCC27853, and mcr-1-harboring E. aerogenes. For the 63 strains, colistin MICs were determined by broth micro-dilution (BMD) using colistin sulfate salt (Sigma-Aldrich, St. Louis, USA) and polystyrene 96-well plates. MIC and disk diffusion were performed according to the 2014 and 2018 CLSI guidelines, and were performed on the same date to prevent human error or contamination. After determination of MICs, experiments were performed for each strain using an appropriate agar concentration. More specifically, for the colistin-resistant strain and the colistin-susceptible strain, to show diameters of inhibition zones, which are distinct from each other, around colistin disks therebetween, commercial MHA (having an agar concentration of 17 g/L), MHA (MHA30) with 30% agar (5.1 g/L); and MHA (MHA30P) with 30% agar (5.1 g/L) and 100 μg/ml of protamine, all of which were predetermined, were used.

Distilled water was added to the protamine, and complete dissolution was performed. Then, the resulting solution was added to MHA before autoclaving. A colistin disk diffusion experiment was performed using a 10 mg colistin disk (BD diagnostics) while culturing the strain for 16 to 18 hours at a condition of 35° C. and 5 vol % CO₂. MHA30P is relatively soft as compared with the commercial MHA, and thus care must be taken to prevent the medium from scratching during inoculation. A sterile cotton swab was soaked in 3 ml of 0.5 McFarland suspension.

2. Analysis and Interpretation

Statistical analysis was performed using SPSS 21 (Version 21.0. Armonk, N.Y., USA). The MIC results were used to interpret the disk diffusion results according to CLSI 2018. As a result, it was interpreted that there was no breakpoint in diameter of an inhibition zone. During the analysis, very major errors (VMEs) were defined as false-susceptible results, and major errors (MEs) were defined as false-resistant results.

3. Results

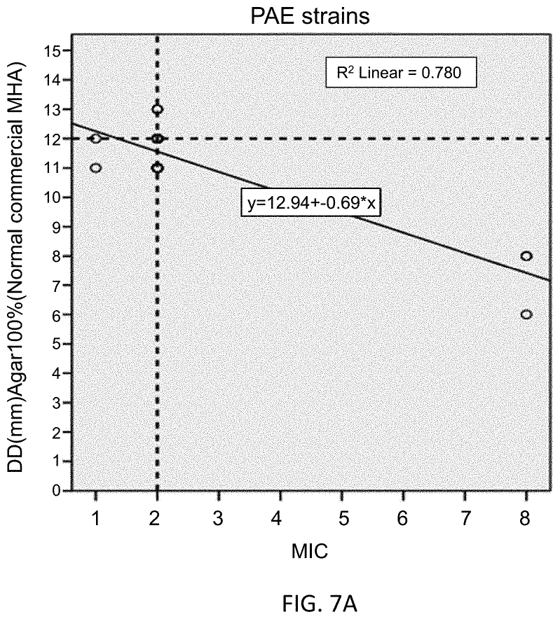

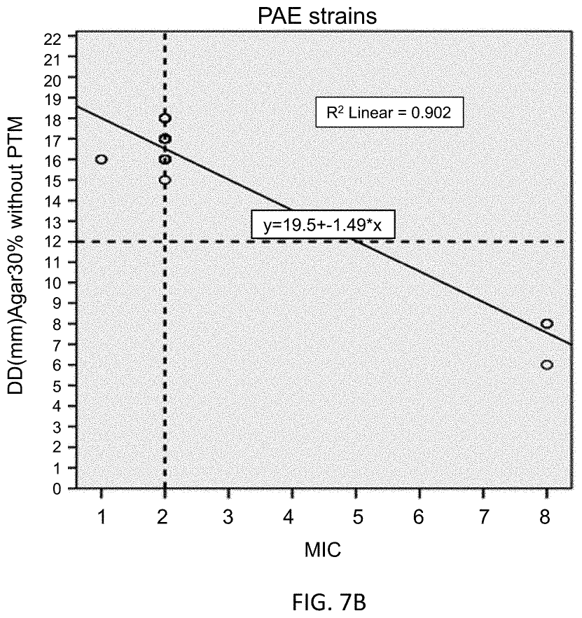

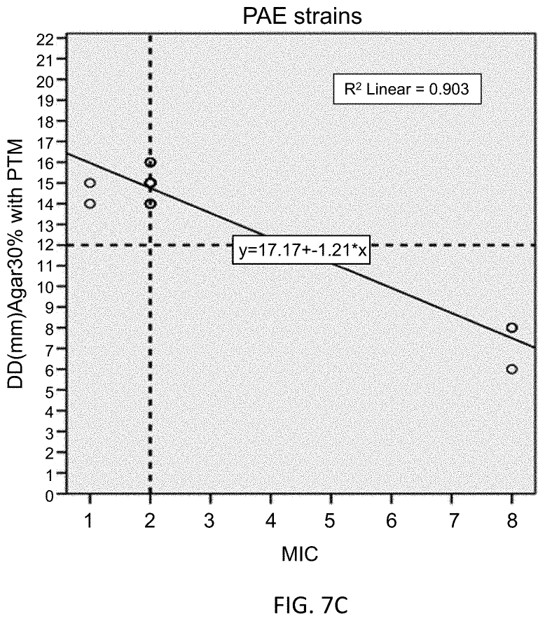

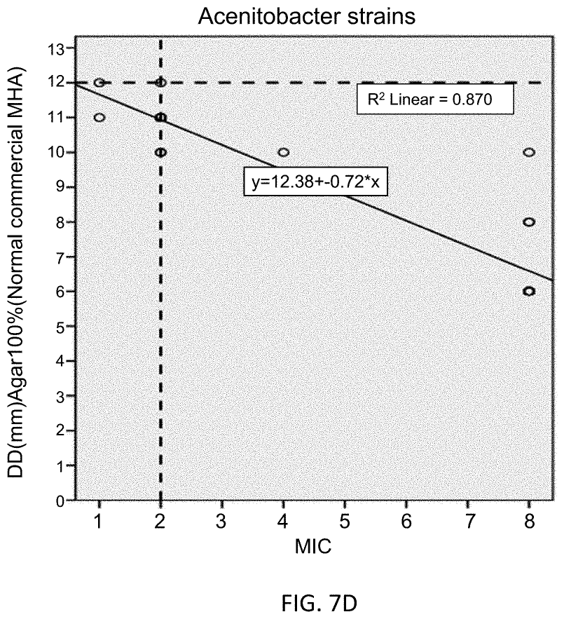

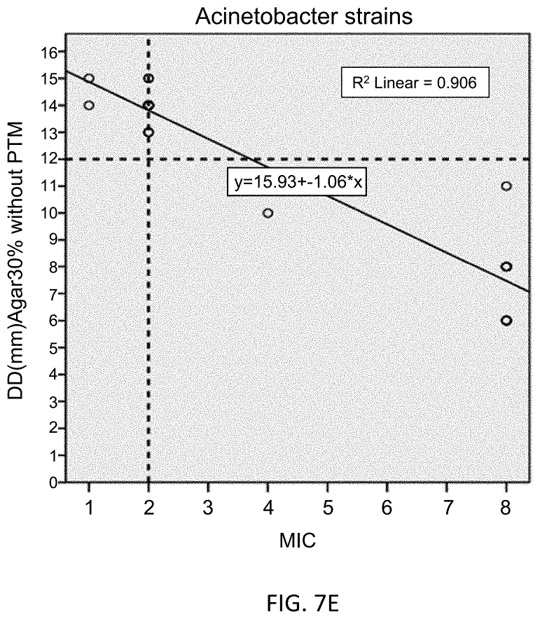

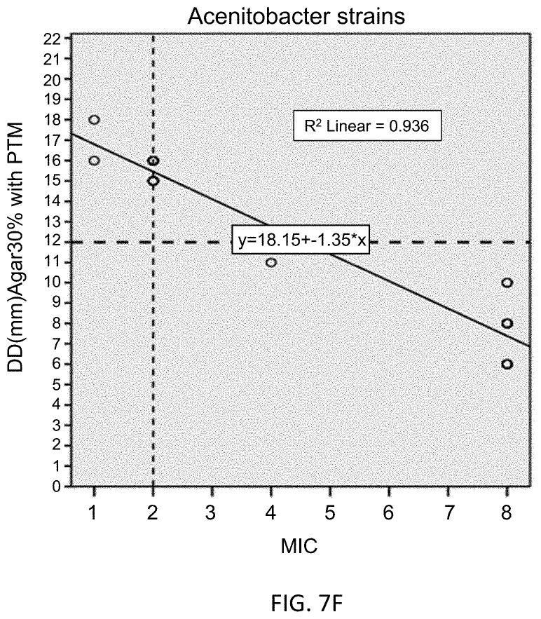

Experiments were performed for colistin diffusion and match rate with MIC using 63-GN clinical isolates, including P. aeruginosa (n=27), ACB (n=33), Escherichia coli ATCC25922, P. aeruginosa ATCC27853, and mcr-1-harboring E. aerogenes. As a result, as illustrated in FIGS. 7A to 7F, for the P. aeruginosa and Acinetobacter strains, it was found that a resistance match rate between MIC and disk diffusion corresponds to 100%. Even for the colistin-susceptible strain, a match rate between MIC and disk diffusion was as high as approximately 97.8%.

In the present invention, in a case of using a medium composition that contains agar at a concentration of 5.1 g/L and protamine at a concentration of 100 μg/ml, diffusion capability of colistin to the agar was increased, and it was possible to identify all colistin-resistant strains such as P. aeruginosa and Acinetobacter spp. From these results, it can be seen that the medium composition can be effectively used to predict colistin resistance. In particular, in P. aeruginosa, a disk diffusion experiment was performed using the medium composition of the present invention. As a result, a 100% match rate with MIC was observed. From this, it can be seen that there are no very major errors or major errors.

### Example 5

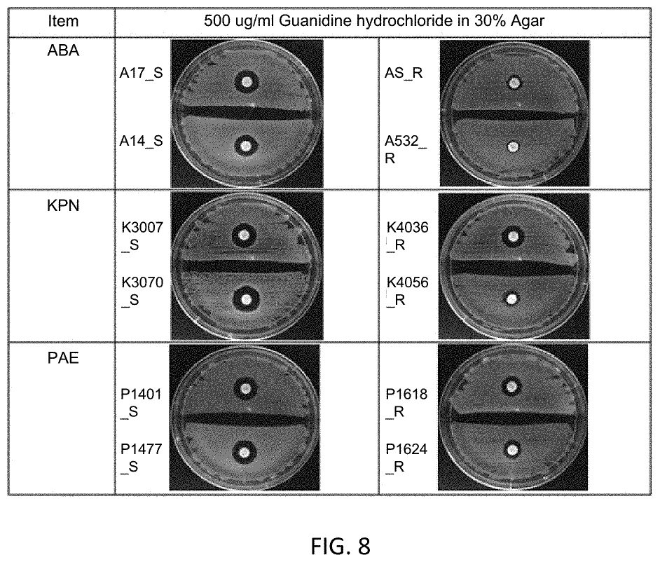

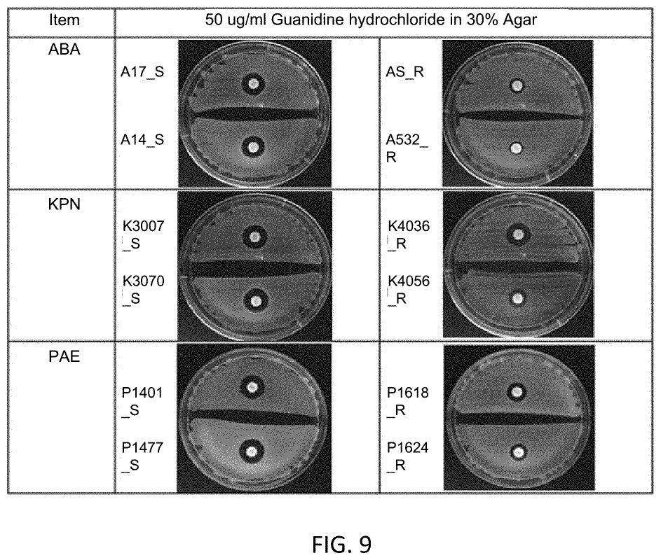

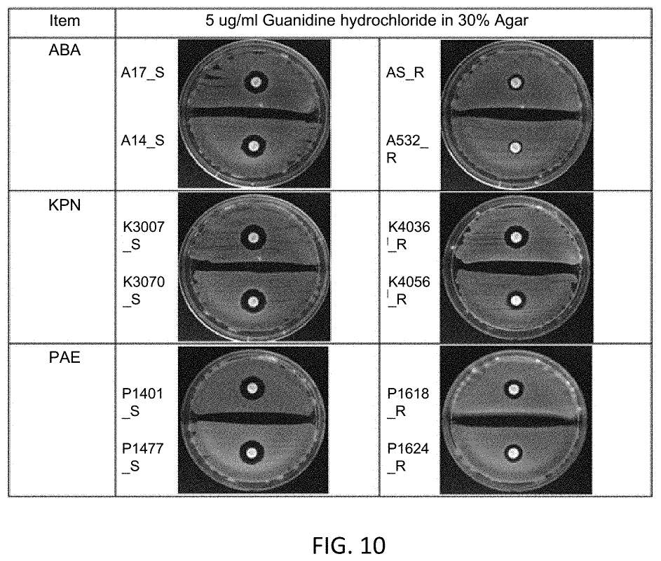

A concentration of agar particles (BD Diagnostic, Sparks, Md., USA) in Mueller-Hinton broth medium (BD Diagnostic) was adjusted to 5.1 g/L, which is 30% of 17 g/L, and guanidine hydrochloride was added to a concentration of 500 μg/ml, 50 μg/ml, or 5 μg/ml. Then, solidification was performed. On a surface of each medium thus prepared was evenly inoculated each of colistin-susceptible (S) or -resistant (R) Acinetobacter baumannii (ABA) strain, colistin-susceptible (S) or -resistant (R) Klebsiella pnemoniae (KPN) strain, and colistin-susceptible (S) or -resistant (R) Pseudomonas aeruginosa (PAE) strain, and a colistin disk was placed thereon. Then, pictures of a growth inhibition zone, which had been formed clear, around the disk were taken for each strain, and the results are illustrated in FIGS. 8 to 10. A diameter of the growth inhibition zone was measured, and the results are shown in Table 3 below.

TABLE 3

| | Guani- | | | | | | | | | | | | |
|---|---|---|---|---|---|---|---|---|---|---|---|---|---|
| | dine | | | | | | | | | | | | |
| | hydro- | | | | | | | | | | | | |
| Agar | chloride | Disk diffusion (mm) | | | | | | | | | | | |
| concen- | concen- | ABA | | | | KPN | | | | PAE | | | |
| tration | tration | A17_S | A14_S | A5_R | A532_R | K3007_S | K3070_S | K4036_R | K4056_R | P1401_S | P1477_S | P1618_R | P1624_R |
| 30% | 500 | 15 | 15 | 10 | 9 | 16 | 17 | 14 | 10 | 16 | 14 | 13 | 12 |
| | μg/ml | | | | | | | | | | | | |
| | 50 | 15 | 15 | 10 | 9 | 16 | 16 | 16 | 12 | 15 | 16 | 13 | 12 |
| | μg/ml | | | | | | | | | | | | |
| | 5 | 15 | 15 | 10 | 9 | 17 | 17 | 16 | 12 | 16 | 16 | 12 | 12 |
| | μg/ml | | | | | | | | | | | | |

As shown in Table 3 and FIGS. 8 to 10, it was found that in a case where the agar concentration was adjusted to 5.1 g/L, which is equivalent to 30% of an agar concentration (17 g/L) in normal Mueller-Hinton medium, and the guanidine hydrochloride was added in an amount of 5 to 500 μg/ml, no changes in size of the growth inhibition zone were observed in the colistin-resistant strains, and the diameter of the growth inhibition zone in the colistin-susceptible strains was increased. In particular, it was found that in a case where the guanidine hydrochloride was added in an amount of 5 to 50 μg/ml, growth of the strain was also not inhibited.

### INDUSTRIAL APPLICABILITY

The present invention relates to a medium composition that can be used in a disk diffusion method to evaluate antibiotic susceptibility, and to a method for evaluating antibiotic susceptibility by a disk diffusion method using the same.

1. A medium composition for a disk diffusion method, comprising:
1.5 to 10 g/L of agar; and
at least one of 50 to 200 μg/ml of protamine or a salt thereof and 5 to 500 μg/ml of guanidine or a salt thereof.

2. The medium composition according to claim 1,
wherein the medium composition is medium for a disc diffusion method for evaluating antibiotic susceptibility.

3. The medium composition according to claim 2,
wherein the antibiotic is a polymyxin.

4. The medium composition according to claim 2,
wherein the antibiotic is colistin.

5. The medium composition according to claim 1,
wherein the salt of protamine is protamine sulfate.

6. The medium composition according to claim 1,
wherein the salt of guanidine salt is guanidine hydrochloride.

7. The medium composition according to claim 1, further comprising:
a beef extract in an amount of 1.5 to 2.5 g/L.

8. The medium composition according to claim 1, further comprising:
a casein hydrolysate in an amount of 15 to 20 g/L.

9. The medium composition according to claim 1, further comprising:
starch in an amount of 1 to 2 g/L.

10. The medium composition according to claim 1,
wherein the medium composition has a pH of 7 to 7.5 at room temperature of 25° C.

11. A method for evaluating antibiotic susceptibility, comprising steps of:
plating a desired strain on the medium composition according to claim 1; and
placing an antibiotic-containing specimen on the strain-plated medium composition.

12. The method according to claim 11,
wherein the specimen is made of paper or cellulose material and is in a disk shape.

13. The method according to claim 11,
wherein the specimen is immersed in an antibiotic-containing solution.

14. The method according to claim 11, further comprising:
after the step of placing the specimen on the medium composition, a step of performing pre-diffusion of the antibiotic at 4° C. for 1 minute to 24 hours.

15. The method according to claim 11, further comprising:
after the step of plating the desired strain, a step of culturing the strain at 5 vol % CO₂ and 37° C. for 10 minutes or longer.

16. The method according to claim 11, further comprising:
after placing the specimen on the medium composition, a step of measuring a size of a microbial growth inhibition zone formed around the specimen, to determine antibiotic susceptibility of the desired strain.

17. The method according to claim 11,
wherein the antibiotic is a polymyxin.

18. The method according to claim 11,
wherein the antibiotic is colistin.
